(19) United States
(12) Patent Application Publication
(10) Pub. No.: US 2019/0385696 A1
(43) Pub. Date: Dec. 19, 2019

(54) METHOD FOR PREDICTING DISEASE RISK BASED ON ANALYSIS OF COMPLEX GENETIC INFORMATION

(71) Applicant: Korea Advanced Institute of Science and Technology, Daejeon (KR)

(72) Inventors: DongHo CHO, Daejeon (KR); Hyein SEO, Daejeon (KR); YongJoon SONG, Daejeon (KR); GyuBum HAN, Daejeon (KR); Dong Jin Ji, Daejeon (KR)

(21) Appl. No.: 16/428,715

(22) Filed: May 31, 2019

(30) Foreign Application Priority Data
May 31, 2018 (KR) 10-2018-0062331
May 31, 2019 (KR) 10-2019-0064200

(51) Int. Cl. G16B 5/20; G16B 5/30; G16B 35/10; G16B 40/00

(52) U.S. Cl. CPC G16B 5/20; G16B 40/00; G16B 35/10; G16B 5/30

(57) ABSTRACT

Provided is a method for diagnosing a disease risk based on complex genetic information network analysis. In the method for diagnosing a disease risk based on complex genetic information network analysis according to the present invention, it is possible to deduce a stable correlation with a disease from a small number of genetic information combination by introducing an optimization method or learning method, and it is possible to provide a genetic information correlation based on a network model. A diagnosis technology satisfying accuracy and economical efficiency enough to be commercially used in an actual medical field by using the correlation between the genetic information and the disease deduced in the present invention will be secured. Further, the biomarker deduced in the present invention will be commercially used in manufacturing a medical device including a diagnosis chip and terminal and in disease diagnosis service.

### CROSS-REFERENCE TO RELATED APPLICATIONS

This application claims priority under 35 U.S.C. § 119 to Korean Patent Application No. 10-2018-0062331, filed on May 31, 2018 and Korean Patent Application No. 10-2019-0064200, filed on May 31, 2019, in the Korean Intellectual Property Office, the disclosure of which is incorporated herein by reference in its entirety.

### TECHNICAL FIELD

The following disclosure relates to a method for diagnosing a disease risk based on complex genetic information network analysis.

### BACKGROUND

As a technical trend for diagnosis of diseases up to now, research is underway to detect genes associated with specific diseases and study functions of genes by using human-shared polymorphism (single nucleotide polymorphism, copy-number variations, base insertion/deletion, or the like) of specific genes or expression information of whole genetic communities to measure changes in expression of genes and proteins using microarrays, protein chips, and the like.

However, the existing studies have focused on one kind of specimen and disease to investigate a relation between the specimen and the disease, such that there is a lack of understanding of the relations and correlation between various genetic information and diseases. Further, there was a problem in that due to a lack of technology for analyzing a relation between complex genetic information and a disease, it was difficult to find a mutation specific to a novel disease that has been undisclosed, and accuracy of a diagnosis method was also significantly low.

A technology for extracting a biomarker from genetic information is a method for statistically analyzing genetic information associated with a disease to extract a marker. However, in the technology for extracting a biomarker according to the related art is performed only within an existing information range obtained by a bottom-up approach, and is still at a level at which a marker is extracted based on partial genetic information including genes, and there is a limitation in that a relation between one piece of genetic information and a disease is one to one.

Further, in a disease diagnosis service based on a biomarker, a method for calculating a contribution degree of specific genetic information to a disease and a character to perform a diagnosis service is used. However, the disease diagnosis service according to the related art has a problem in that the disease diagnosis service depends on deducing a simple relation between one kind of disease and one kind of genetic information and does not perform complex analysis between diseases and genetic information, and has a limitation in that it is impossible to reflect changes in characteristics depending on high-dimensional variables such as passage of time, treatment, recurrence, and the like, as additional variables. Therefore, there is a limitation in that accuracy of the diagnosis is low, and a different result is deduced depending on the kind of service platform.

### RELATED ART DOCUMENT

[Patent Document]

(Patent Document 1) WO 2014-052909

### SUMMARY

An embodiment of the present invention is directed to providing a biomarker for diagnosing a disease and a method for predicting a disease risk by deducing disease state-specific information from relations between complex genetic information and utilizing optimization based on a network model and a machine learning method based on artificial intelligence.

Another embodiment of the present invention is directed to providing a biomarker having excellent accuracy of diagnosis and excellent economical efficiency by analyzing relations between genetic information for understanding relations between complex and various genetic information and diseases and extracting an optimized genetic information combination to introduce an analysis method based on a network model.

In one general aspect, there is provided a method for predicting a disease risk based on complex genetic information network analysis including:

extracting complex genetic information from specimens of a disease patient and a normal person;

comparing and analyzing the complex genetic information network to construct a complex genetic information library;

applying an optimization method or learning method to the complex genetic information library to deduce a disease state-specific biomarker; and

constructing a network model for predicting a disease risk from the disease state-specific biomarker and predicting a risk.

In another general aspect, there is provided a disease state-specific biomarker deduced by the method for predicting a disease risk based on complex genetic information network analysis described above.

### BRIEF DESCRIPTION OF THE DRAWINGS

FIG. 1 shows an example of a concept of a method for diagnosing a disease risk based on complex genetic information network analysis according to the present invention.

FIG. 2 shows a concept of step-by step genetic information based on a gene expression process.

FIG. 3 shows an example of a concept of a method for deducing and validating a disease state-specific biomarker using a learning method.

FIG. 4 shows an example of a characteristic modeling of a disease state-specific biomarker.

FIG. 5 shows an example of a convolutional neural network (CNN) analysis method with respect to protein expression data.

FIG. 6 shows an example of an algorithm for predicting a digestive cancer risk from mi-RNA information.

FIG. 7 shows a result of validation using only basic CNN analysis.

FIG. 8 shows a result of a change in a result after extracting and learning important mi-RNA candidate combinations.

FIG. 9 shows a result of a possibility of simultaneous screening and precise diagnosis confirmed in proteins.

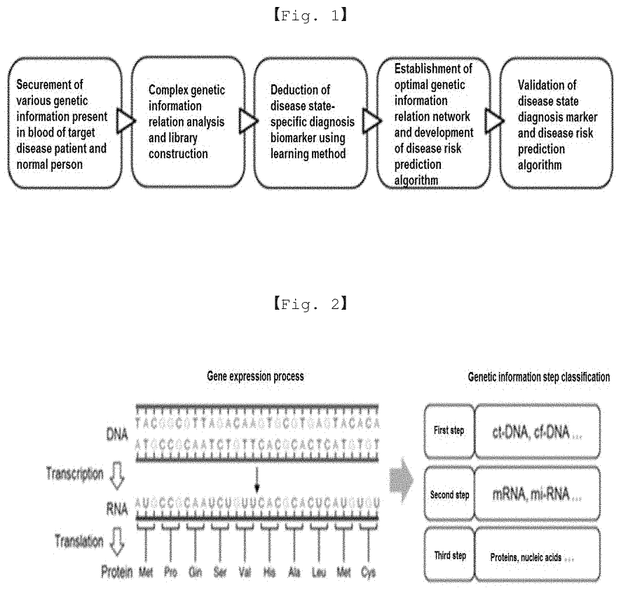

### DETAILED DESCRIPTION OF EMBODIMENTS

Hereinafter, a method for diagnosing a disease risk based on complex genetic information network analysis according to the present invention will be described in detail with reference to accompanying tables or drawings.

The drawings to be provided below are provided by way of example so that the spirit of the present invention can be sufficiently transferred to those skilled in the art. Therefore, the present invention is not limited to the accompanying drawings provided below, but may be modified in many different forms. In addition, the accompanying drawings suggested below will be exaggerated in order to clear the spirit and scope of the present invention.

Technical terms and scientific terms used in the present specification have the general meaning understood by those skilled in the art to which the present invention pertains unless otherwise defined, and a description for the known function and configuration unnecessarily obscuring the gist of the present invention will be omitted in the following description and the accompanying drawings.

In the present invention, the term “specimen sample” or “sample” means genetic information secured for analysis and is used as the same meaning throughout the present specification.

The present invention relates to a method for diagnosing a disease risk based on analysis of a complex genetic information network in the blood.

According to the present invention, a disease state-specific biomarker, which assists in understanding functions of genetic information by comparing, analyzing, and determining general biological phenomena and disease-associated information based on the extracted complex genetic information, and additionally has high accuracy, may be deduced, and a model for predicting a disease risk may be constructed.

In the present invention, in order to deduce the disease state-specific biomarker and construct the model for predicting a disease risk, a big data processing method, a deep learning method based on artificial intelligence, for example, a machine learning method, or the like, may be used in combination with a vast amount of genetic information.

Hereinafter, the method for predicting a disease risk based on analysis of complex genetic information will be described in detail.

The present invention provides a method for predicting a disease risk based on complex genetic information network analysis, the method including:

extracting complex genetic information from specimens of a disease patient and a normal person;

comparing and analyzing the complex genetic information network to construct a complex genetic information library;

applying an optimization method or learning method to the complex genetic information library to deduce a disease state-specific biomarker; and

constructing a network model for predicting a disease risk from the disease state-specific biomarker and predicting a risk.

Hereinafter, each process will be described in detail.

First, the extracting of the complex genetic information from the specimens of the disease patient and the normal person will be described in detail.

In the extracting of the complex genetic information from the specimens of the disease patient and the normal person, information associated with DNA, RNA, protein, or the like with respect to the entire genome of the specimens may be secured. A method for acquiring the information is not limited as long as the object of the present invention is not hindered. As an example, the information may be secured from a genetic information database, or the like. As a more specific example, a database provided by National Institutes of Health (NIH) may be used, and as a more specific example, information associated with cancer may be secured through the entire genetic information depending on the kind of cancer provided by the Cancer Genome Atlas (TCGA). As another example, information may be obtained by analyzing the genome sequencing of a specimen sample taken in a hospital or directly taken from a patient. As another example, a whole exome sequence set performing a direct role in synthesizing protein in genes may be secured and used, but the method for acquiring the information is not limited thereto.

In the present invention, genome sequence information of the specimen may be partially changed depending on the kind of genetic information database, a device used in the sequencing, a sequencing method, or the like. Further, the genome sequence information is not limited as long as the object of the present invention is not hindered. For example, the genome sequence information is based on information provided in a human genome map identified in a human genome project.

In the present invention, whole genome sequence information of the specimens of the disease patient and the normal person may become basic information in detecting another biomarker in the present invention, and analysis is performed on the basis of a difference in the genome sequence information of the specimen including DNA information such as cf-DNA and ct-DNA, RNA expression information of mRNA, mi-RNA, or the like, protein synthesis information, and the like, which may be obtained from the genome sequence information as described above. Among the whole genome sequence information, although not limited, chromosome information, information associated with a position of a nucleotide sequence in the chromosome, nucleotide sequence variation information associated with insertion, deletion, or substitution of the nucleotide sequence, RNA information, protein expression information, information including a three-dimensional structure of a protein and reliability, and the like, may be mainly used to detect a biomarker for diagnosing a disease.

In the present invention, at the time of analyzing information included in the genome sequence information, the information may be added and deleted depending on the kind, a version, and use environments of a used program.

Next, the comparing and analyzing of the complex genetic information to construct the complex genetic information library will be described in detail.

In the comparing and analyzing of the complex genetic information to construct the complex genetic information library, a complex relation existing in the genetic information obtained in the extracting of the complex genetic information from the specimens of the disease patient and the normal person may be analyzed, such that important genetic information associated with the disease may be extracted and a library thereof may be constructed.

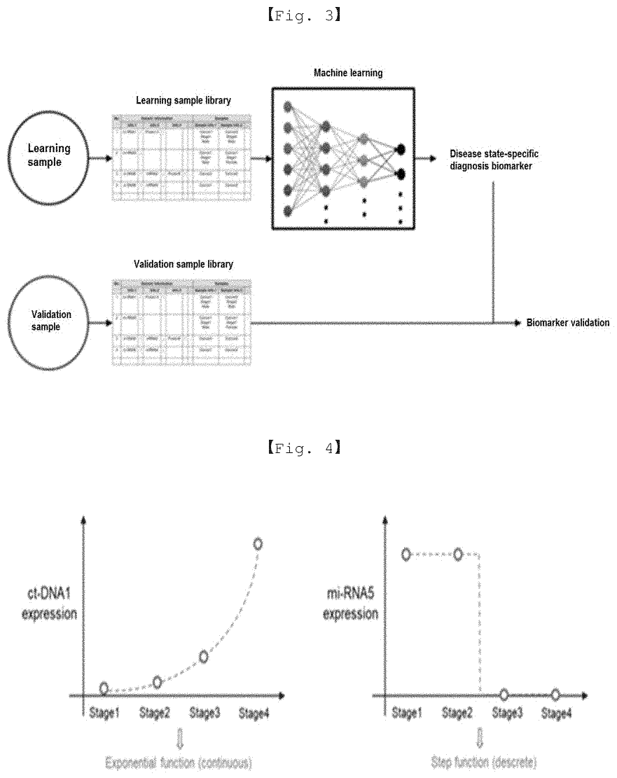

In the present invention, the genetic information is not limited as long as the object of the present invention is not hindered. Examples of the genetic information may include DNA information such as cf-DNA and ct-DNA associated with a gene expression process, RNA expression information of mRNA, mi-RNA, or the like, and protein synthesis information (FIG. 2).

In order to extract an important genetic information factor, which is a target of analysis, although not limited as long as the object of the present invention is not hindered, the following process may be included.

First, it is possible to extract classification accuracy for the case in which a normal group and a disease group may be distinguished using a single genetic information factor. The kind and number of single genetic information factor are not limited as long as the normal group and the disease group may be distinguished from each other only with the information. Examples of the single genetic information factor may include single nucleotide polymorphism (SNP) variations including nucleotide sequence variations associated with addition, deletion, or substitution of a nucleotide sequence, copy-number variations (CNVs), amino acid sequence polymorphism of proteins, and the like, but are not limited thereto. As an example, in the case in which a nucleotide sequence variation is commonly shown in specimen samples of the disease group and there is no nucleotide sequence variation commonly in specimen samples of the normal group, it is preferable to grasp the corresponding genetic information and extract and store position information and variation information of the nucleotide sequence.

Next, it is possible to set an on/off tag capable of determining whether or not the corresponding genetic information factor has an influence on selection of the disease group by measuring a difference between an actual expression amount and a reference amount with respect to each genetic information factor. As an example of the setting method as described above, reference values of expression amounts in respective steps associated with an important genetic gene expression process may be defined as Th₁, Th₂, and Th₃, respectively, and when genetic information expression amounts are increased or decreased due to a disease, increase reference values (Th₁^{up}, Th₂^{up}, and Th₃^{up}) and decrease reference values (Th₁^{down}, Th₂^{down}, Th₃^{down}) may be defined and used, respectively. By using the variable defined as described above, it is possible to extract genetic information which satisfies each expression amount reference and of which an expression amount is changed by a disease with respect to the secured specimen sample. In this case, if necessary, nucleotide sequence information of the corresponding genetic information may be secured and used, and it is possible to extract variants in DNA, RNA and protein sequences such as single nucleotide polymorphism (SNP) variations including nucleotide sequence variations associated with addition, deletion, or substitution of the nucleotide sequence as described above and copy-number variations (CNVs) to utilize nucleotide sequence variation information by the disease, but the present invention is not limited thereto.

It is possible to grasp correlation between complex genetic information by analyzing a change in expression amount between genetic information corresponding to different steps, nucleotide sequence variations, and the like in the steps shown in FIG. 2 to construct the library using the extracted genetic information factor, and the correlation may be utilized to deduce a biomarker later.

TABLE 1
Example of library construction through
genetic information relation analysis
Th₁^{up}, Th₂^{up}, Th₃^{up}, Th₁^{down}, Th₂^{down}, Th₃^{down}

| | Generic Information | | | | | | | Samples | | |
|---|---|---|---|---|---|---|---|---|---|---|
| No. | Info. 1 | | Info. 2 | | Info. 3 | | . . . | Sample Info. 1 | Sample Info. 2 | . . . |
| 1 | mi-RNA1 | Increase | Protein 5 | Increase | | | . . . | Gastric Cancer | Liver Cancer | . . . |
| | | | | | | | | Stage 1 | Stage 2 | |
| | | | | | | | | Male | Male | |
| | | | | | | | | . | . | |
| | | | | | | | | . | . | |
| | | | | | | | | . | . | |
| 2 | mi-RNA5 | SNP1 | | | | | . . . | Gastric Cancer | Gastric Cancer | . . . |
| | | | | | | | | Stage 1 | Stage 1 | |
| | | | | | | | | Male | Female | |
| | | | | | | | | . | . | |
| | | | | | | | | . | . | |
| | | | | | | | | . | . | |
| 3 | ct-DNA6 | Increase | mRNA2 | Decrease | Protein9 | Increase | . . . | Liver Cancer | Breast Cancer | . . . |
| | | | | | | | | . | . | |
| | | | | | | | | . | . | |
| | | | | | | | | . | . | |
| 4 | ct-DNA8 | Decrease | miRNA4 | Decrease | | | . . . | Gastric Cancer | Colon Cancer | . . . |
| | | | | | | | | . | . | |
| | | | | | | | | . | | |
| | | | | | | | | . | | |
| . | . | . | . | . | . | . | . . . | . | . | . . . |
| . | . | . | . | . | . | . | | . | . | |
| . | . | . | . | . | . | . | | . | . | |

As an example of library construction through genetic information relation analysis, a library may be constructed by recording genetic information in the case in which it is observed that an expression amount of mi-RNA1 is equal to or less than a reference value Th₂^{down} in a male patient with stage 1 gastric cancer and a male patient with stage 2 liver cancer and at the same time, an expression amount of protein5 is equal to or more than a reference value Th₃^{up} as shown in Table 1; a case in which SNP1 of mi-RNA5 was observed in a male with stage 1 gastric cancer and a female with stage 1 gastric cancer, but a relation with another specific genetic information was not found; and the like.

As an example, the information analyzed by the above-mentioned method may be converted into a predetermined platform, that is, the same frame form as shown in Table 1 to thereby be stored or managed.

Next, the applying of the optimization method or learning method to the complex genetic information library to deduce the disease state-specific biomarker will be described in detail.

In the applying of the optimization method or learning method to the complex genetic information library to deduce the disease state-specific biomarker, the disease state-specific biomarker may be deduced by analyzing the complex genetic information library constructed by the above-mentioned method using the optimization method or the learning method.

A method for extracting a disease state-specific biomarker candidate is not limited as long as the object of the present invention is not hindered, but it is possible to confirm the presence or absence of the same genetic information relation between a specimen sample in a disease state to be confirmed and the library from the deduced complex genetic information library and extract multi-dimensional relations between the increase or decrease of variation information, the genetic information, nucleotide sequence, and the number of genetic information from the confirmed genetic information, such that this extracted relation may be selected as a candidate group for deducing the disease state-specific biomarker. In the selecting of the candidate group, it is preferable that the disease state-specific biomarker simultaneously minimizes the number of genetic information to be considered while significantly increasing accuracy of the corresponding marker indicating a disease state. To this end, after defining the candidate group in a form of multi-variable optimization, the disease state-specific marker may be deduced by applying a mathematical algorithm, but is not limited thereto.

Any mathematical algorithm for multi-variable optimization may be introduced without limitation as long as it may solve problems of multi-variable functions. For example, a simulated annealing method, a genetic algorithm, a tap search method, a simulated evolution method, a probabilistic evolution method, and the like may be mentioned, and preferably, the genetic algorithm may be used. In the case of extracting the disease state-specific biomarker through the above-mentioned method, there is no need to necessarily complete the entire process, and the process may be stopped during obtaining an optimal solution, and among the obtained solutions at that time, the most preferable solution may be used.

The genetic algorithm is based on biological genetics of the natural world and is a method to find a better solution by expressing possible solutions to a problem in a form of a predetermined data structure by a parallel and global search algorithm to gradually modify the data structure. Here, the data structure indicating the solutions may be expressed as the genes, and a process of modifying the data structure to gradually make a better solution may be expressed as evolution. In other words, the genetic algorithm may be a simulated evolution search algorithm for finding a solution x optimizing an unknown function Y=f(x). The genetic algorithm is close to an approach method for solving a problem rather than an algorithm for solving a specific problem, and may be applied to all problems that may be modified and expressed in a form capable of being used in the genetic algorithm. Generally, in the case in which the problem is too complicated to be calculated, even though it is impossible to obtain an actual optimal solution, it is possible to approach the solution through the genetic algorithm as a method for obtaining a solution close to an optimal solution, which is preferable.

As an example of a method for deducing the disease state-specific biomarker, a learning sample, which is an analysis target, and a validation sample for validating accuracy of the learning sample may be provided as shown in FIG. 3. As an example, the validation sample includes only the corresponding disease state-specific genetic information through the existing analysis, but is not limited thereto. As performed in Example of the present invention, an analysis target library may be randomly divided into the learning sample and the validation sample, and the learning may be performed. Further, accuracy may be improved by repeating a learning process several times.

Since it is difficult to calculate classification accuracy with respect to all subsets and complexity is increased in the case in which a size of the library is large, it is preferable to perform a process for decreasing complexity. When the size of the library is N, the number of all subsets is 2\hat{ }N. Therefore, in the case in which the size of the library is large, since it is difficult to calculate classification accuracy with respect to all subsets and complexity is increased, in order to solve this problem, there is a need to decrease complexity, for example, using a heuristic algorithm, or the like. As an example, when the size of a subset is N, in the case of confirming a possibility of a marker and decreasing a size of a set step by step by preferentially considering only the case in which the possibility is largest, the entire number of cases for a marker to be investigated is decreased to N(N+1)/2.

Selection of the genetic information, which is a variable for multi-variable optimization, is not limited as long as the object of the present invention is not hindered. For example, genetic information may be randomly selected according to the heuristic algorithm, and preferably, a combination of genetic information having the maximum accuracy may be selected. As an example, when there is a characteristic that genetic information mi-RNA1 and ct-DNA5 are simultaneously increased, information associated with increases and decreases in respective expression amounts of mi-RNA1 and ct-DNA5 may be utilized in the learning and used as two features, respectively, and whether or not the characteristic that genetic information mi-RNA1 and ct-DNA5 are simultaneously increased is present in a sample may be used as one feature in the learning.

In the present invention, the kind of learning method based on artificial intelligence used in the learning for deducing the biomarker is not limited as long as the object of the present invention is not hindered. As an example, a neural network, a deep learning method, or the like, may be used. As an example of the neural network, convolutional neural network (CNN), a recurrent neural network (RNN), or the like, may be mentioned. As an example, CNN may be used as in Example of the present invention, but the learning method is not limited thereto, and a suitable learning method may be selected and used depending on the secured data and features of the biomarker.

In the present invention, preferably, a process for validating performance of the disease state-specific biomarker deduced by the above-mentioned method may be further performed. To this end, by calculating classification accuracy after applying the deduced disease state-specific biomarker to a sample that is not used to detect the biomarker or a normal sample, accuracy of the deduced biomarker may be validated, which is more preferable.

Next, the constructing of the network model for predicting a disease risk from the disease state-specific biomarker and the predicting of the risk will be described in detail.

In the constructing of the network model for predicting a disease risk from the disease state-specific biomarker, state changes such as occurrence, progression, recurrence of a disease, and the like, may be constructed in a form of a network from the disease state-specific biomarker deduced using the complex genetic information library obtained by analyzing the relation between the complex genetic information and the optimization method or learning method.

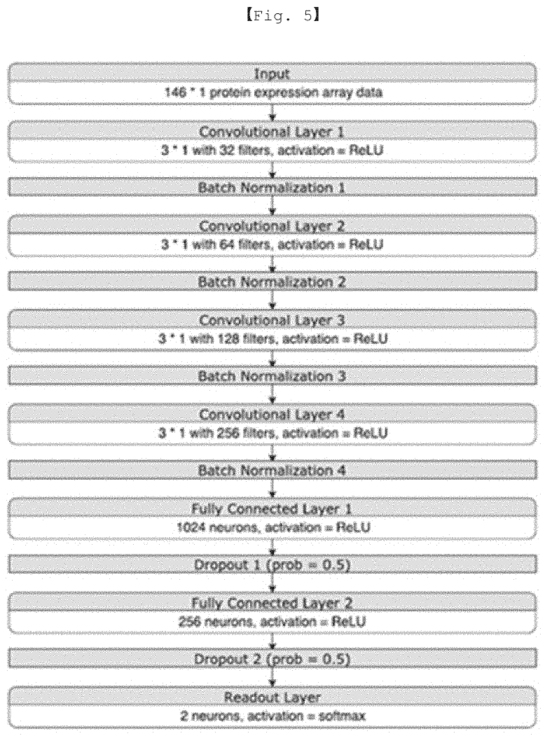

A method for constructing the network is not limited as long as the object of the present invention is not hindered, but the method may include a method for analyzing an information change in the disease state-specific biomarker deduced according to a specific disease state change using the genetic information library constructed by the above-mentioned method. As an example of analysis, discontinuous expression changes of ct-DNA1 or mi-RNA5, which is genetic information, may be tracked and modeled in a form of a mathematical function as shown in FIG. 4. A form of the mathematical function is not particularly limited, but as an example, it is preferable to select a regression function capable of approximately satisfying data of the discontinuous expression change.

A regression analysis method used to constitute the regression function is classified into simple regression analysis and multiple regression analysis, wherein the simple regression analysis may be used to analyze a relation between a single dependent variable and a single independent variable, and the multiple regression analysis may be used to find out a relation between a single dependent variable and several independent variables. In the case of an expression exchange shown by way of example in FIG. 4, a regression function may be obtained by simple regression analysis using a single dependent variable and a single independent variable, respectively. As an example, expression of ct-DNA1 in FIG. 4 may be modeled as an exponential function, and expression of mi-RNA5 may be modeled as a regression function in a form of a step function.

After mathematically modeling features of the disease state-specific biomarker through the above-mentioned method, a genetic information relation network model, which is a network model for predicting a disease risk, composed of genetic information, may be established so as to track a changing process of genetic information depending on main state changes in the disease.

A form of the genetic information relation network model is not limited as long as the object of the present invention is not hindered. The genetic information relation network model may be in a form of a static disease network to which only correlation between complex genetic information is applied or a dynamic disease network to which the passage of time or individual-specific genetic information such as habits, or the like, is additionally added as a variable. Preferably, the genetic information relation network model may be in a form of the dynamic disease network. It is possible to tract genetic information characteristics continuously changed and to diagnose and predict a disease by using the network model in the above-mentioned form, which is preferable.

In the present invention, accuracy of the biomarker and the genetic information relation network model, which is a network model for predicting a disease risk is not limited, but may be evaluated using the following indicators.
Sensitivity: This is a measurement indicator for evaluating whether or not a patient with an actual disease is satisfactorily classified, and may be defined as TP/(TP+FN) for preventing diagnosis failure based on misdiagnosis, wherein TP is the number of cases in which a patient with a disease is classified as a disease patient, and FN is the number of cases in which a patent with a disease is classified as a normal person. When sensitivity of the biomarker and the network model for predicting a disease risk is preferably 95% or more, more preferably 99% or more, and most preferably 99.9% or more, an examination cost may be decreased, a commercialization possibility may be increased, and the case in which a disease risk may be confirmed by one-time examination using main genetic information associated with a large number of diseases is increased, which is preferable.
Specificity: This is a measurement indicator for evaluating whether or not a normal person is satisfactorily classified and is defined as a TN/(TN+FP) in order to prevent unnecessary follow-up examination caused by false disease diagnosis, wherein TN is the number of cases in which a normal person is classified as a normal person, and FP is the number of cases in which the normal person is classified as a patient with a disease. When specificity of the biomarker and the network model for predicting a disease risk is preferably 90% or more, more preferably 95% or more, and most preferably 99% or more, an examination cost may be decreased, a commercialization possibility may be increased, and the case in which a disease risk may be confirmed by one-time examination using main genetic information associated with a large number of diseases is increased, which is preferable.

In predicting a disease risk, sensitivity or specificity may be used alone or in combination, and among them, sensitivity is more important in predicting a disease risk than specificity, such that it is more preferable to use sensitivity together with specificity.

In the present invention, any disease may be applied as long as the biomarker may be deduced. For example, the disease may be a disease requiring rapid diagnosis such as cancer. As a more specific example, the cancer may be one or more selected from the group consisting of bladder urothelial carcinoma, breast invasive carcinoma, cervical and endocervical cancers, colon cancer, colon adenocarcinoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thyroid carcinoma, thymoma, and uterine corpus endometrial carcinoma, preferably, one or more selected from the group consisting of bladder urothelial carcinoma, breast invasive carcinoma, colon cancer, colon adenocarcinoma, cervical and endocervical cancers, liver hepatocellular carcinoma, lung adenocarcinoma, kidney chromophobe, kidney renal clear cell carcinoma, kidney renal papillary cell carcinoma, ovarian serous cystadenocarcinoma, prostate adenocarcinoma, lung squamous cell carcinoma, and stomach adenocarcinma, and more preferably, one or more selected from the group consisting of breast invasive carcinoma, colon cancer, and stomach adenocarcinoma, but is not limited thereto.

In addition, the present invention provides a disease state-specific biomarker deduced by the method for predicting a disease risk through the complex genetic information network analysis.

The biomarker deduced in the present invention may be expected to be commercially used in manufacturing a medical device including a diagnosis chip and terminal and in disease diagnosis service to thereby be efficiently used in determining prognosis of a disease, and the like.

Hereinafter, the contents of the present invention will be described in more detail through Examples. Examples are to describe the present invention in more detail, and the scope of the present invention is not limited thereto.

[Experimental Materials]

1. The following mi-RNA related data were secured and used.

1) GSE54397

Data provided from a research database by Professor Nayoung Kim of Seoul National University were used.

Among microarray data of normal tissue and cancer tissue samples of 16 gastric cancer patients in the database, 3523 kinds of mi-RNA data were provided and used.

2) GSE61741

Data provided from a database of Saarland university was used.

Among microarray data of blood samples of a total of 1049 persons including cancer patients and normal persons in the database, a total of 848 kinds of mi-RNA data of a total of 136 samples obtained from 13 gastric cancer patients, 29 colon cancer patients, and 94 normal persons were provided and used.

3) TCGA NGS data

Data were provided from a TCGA database and used.

In the database, 45 normal tissue samples and 446 cancer tissue samples of gastric cancer patients were downloaded, respectively, and mi-RNA and NGS read information were secured.

A total of 211 kinds of mi-RNA data were used.

2. The following data other than mi-RNA were secured and used.

1) TCGA protein expression array data

This is a protein expression database for patients with breast cancer, thyroid cancer, liver cancer, kidney cancer, and lung cancer and normal persons.

From the database, breast cancer disease, breast cancer normal, thyroid cancer, liver cancer, kidney cancer 1 (kidney renal clear cell carcinoma), kidney cancer 2 (kidney renal papillary cell carcinoma), kidney cancer 3 (kidney chromophobe), lung cancer 1 (lung adenocarcinoma), and lung cancer 2 (lung squamous cell carcinoma) data were secured,

wherein the number of respective samples were 1078, 45, 426, 183, 478, 215, 63, 365, and 327, respectively.

Only a total of about 200 kinds of protein expression amount data were present per each sample, and only 146 protein data commonly present in all the samples were extracted and used.

TABLE 2
Examples of mi-RNA and protein expression
data

| ID_REF | GSM1314 [symbol] | GSM1314 [symbol] | GSM1314 [symbol] | GSM1314 [symbol] | GSM1314 [symbol] | GSM1314 [symbol] | GSM1314 [symbol] | GSM1314 [symbol] | [symbol] |
|---|---|---|---|---|---|---|---|---|---|
| A_25_P00 [symbol] | 2.97188 | 4.271208 | 3.448411 | 2.076416 | 0.715718 | −0.4122 | 25.73534 | 6.101282 | |
| A_25_P00 [symbol] | −1.04147 | 0.721858 | 20.94287 | −0.10715 | 0.610717 | −0.83561 | 2.006146 | 1.308552 | |
| A_25_P00 [symbol] | −0.85218 | 1.189662 | −0.96096 | −0.64335 | −0.29708 | −1.10413 | −1.68779 | −0.09533 | |
| A_25_P00 [symbol] | 0.77163 | 3.635078 | 3.382378 | 3.152691 | 1.529792 | 0.091025 | 25.07302 | 1.561757 | |
| A_25_P00 [symbol] | −1.09483 | 0.130037 | −0.15125 | −0.38508 | −0.83953 | −0.31306 | 0.214257 | −1.61005 | |
| A_25_P00 [symbol] | −1.42786 | −1.15086 | −1.30777 | −1.74881 | 0.017548 | −0.42399 | −2.42501 | −0.53879 | |
| A_25_P00 [symbol] | −1.93991 | −0.89703 | −1.10407 | −1.49793 | −1.07525 | −1.23038 | −1.88413 | −1.34954 | |
| A_25_P00 [symbol] | −1.87056 | 0.151268 | −1.73956 | −1.03857 | −0.20014 | −1.6963 | 37.41247 | −1.33654 | |

[symbol]

indicates data missing or illegible when filed

### [Example 1] Application of Learning Method to Protein Data

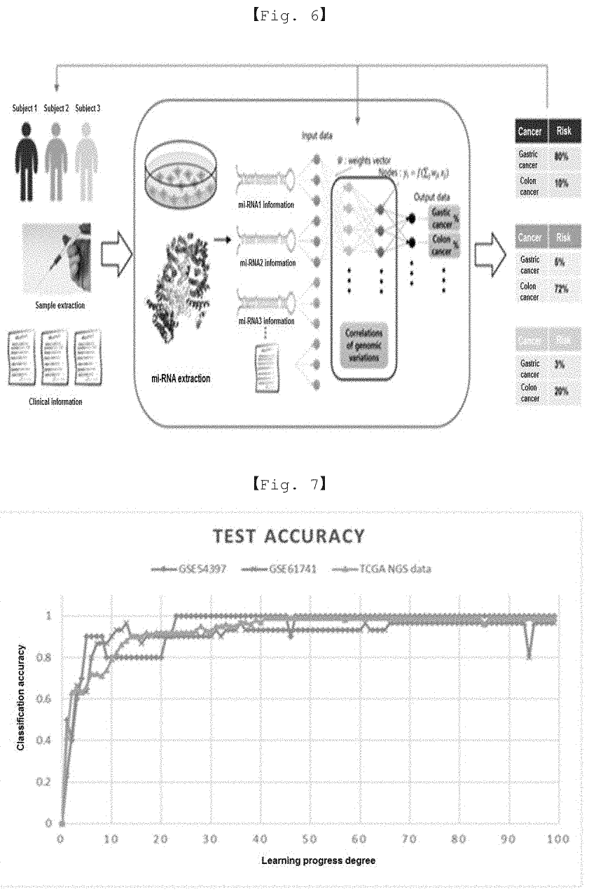

A relationship between the protein data and input data was deduced using a CNN method in FIG. 5, passed through a fully connected layer, and then, finally classified using a softmax.

### [Example 2] Accuracy Prediction

The learning progressed by applying the same CNN network method as in Example 1.

1. GSE54397

The learning progressed using 22 samples among 32 samples, and validation was performed using the other 10 samples.

2. GSE61741

The learning progressed using 106 samples among 136 samples, and validation was performed using the other 30 samples.

3. TCGA NGS data

The learning progressed using 391 samples among 491 samples, and validation was performed using the other 100 samples.

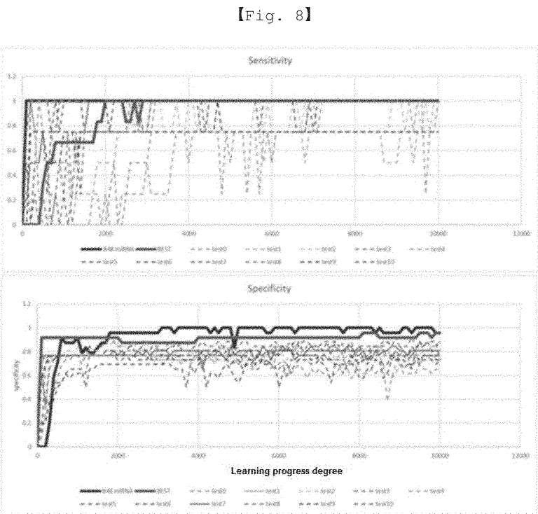

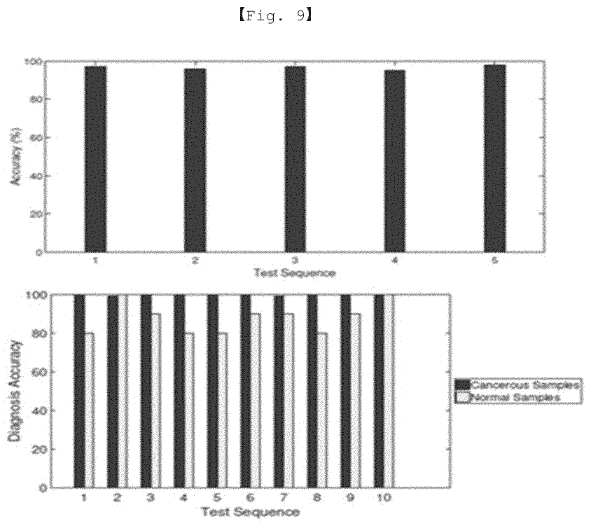

The validation results are shown in FIGS. 7 and 8.

Referring to the result in FIG. 7, it was confirmed that as the learning progressed, classification accuracy in the GSE54397 model (tissue, microarray data) was 100%, classification accuracy in the GSE61741 (blood, microarray data) was about 96.67%, and classification accuracy in the TCGA NGS data was about 99%, and thus, significantly high accuracy of 95% or more was exhibited in all cases.

Referring to FIG. 8, it was confirmed that as an extraction process of important mi-RNA progressed, in all the cases of extraction of 848 mi-RNAs and 30 optimal mi-RNAs (BEST), sensitivity approached 1 as the learning progressed. Further, in view of specificity, in the cases of extraction of 848 mi-RNAs, fluctuation in the vicinity of 1 was shown as the learning progressed, and in the cases of extraction of 30 optimal mi-RNAs (BEST), the specificity approached to 0.95 or more.

### [Example 3] Deduction of Biomarker Using Clinical Data

Data on three diseases of breast cancer, gastric cancer, and colon cancer were all secured from a database of The Cancer Genome Atlas (TCGA) project being conducted by NIH in the United States since 2006. Specific database names used to secure respective disease data were as follows.

Breast cancer: TCGA-BRCA, Gastric cancer: TCGA-STAD, and colon cancer: TCGA-COAD.

30 kinds of biomarkers were deduced for each type of cancer by performing the learning through a CNN network method on mi-RNA genetic information data among them using the same method as in Example 1.

The results are as shown in the following Table 3.

TABLE 3
Optimal mi-RNA biomarker (BEST)
depending on the kind of cancer

| Kind of | |
|---|---|
| Cancer | mi-RNA Biomarker |
| Breast | ‘hsa-mir-30d’, ‘hsa-mir-145’, ‘hsa-mir-425’, |
| Cancer | ‘hsa-mir-203a’, ‘hsa-mir-452’, ‘hsa-mir-378a’, |
| | ‘hsa-mir-455’, ‘hsa-mir-100’, ‘hsa-mir-199b’, |
| | ‘hsa-mir-205’, ‘hsa-mir-542’, ‘hsa-mir- |
| | 532’, ‘hsa-mir-625’, ‘hsa-mir-200c’, ‘hsa-mir- |
| | 183’, ‘hsa-mir-22’, ‘hsa-mir-451a’, ‘hsa-mir- |
| | 30a’, ‘hsa-mir-30e’, ‘hsa-mir-148a’, ‘hsa-mir- |
| | 143’, ‘hsa-mir-375’, ‘hsa-mir-584’, ‘hsa-mir- |
| | 379’, ‘hsa-mir-10a’, ‘hsa-mir-182’, ‘hsa-mir- |
| | 21’, ‘hsa-mir-486-1’, ‘hsa-mir-486-2’, ‘hsa-mir- |
| | 10b’ |
| Colon | ‘hsa-mir-6086’, ‘hsa-mir-3118-1’, ‘hsa-mir- |
| Cancer | 1321’, ‘hsa-mir-548f-5’, hsa-let-7c’, ‘hsa-mir- |
| | 4752’, ‘hsa-mir-183’, ‘hsa-mir-29a’, ‘hsa-mir- |
| | 30e’, ‘hsa-mir-486-1’, ‘hsa-mir-194-1’, ‘hsa- |
| | mir-194-2’, ‘hsa-mir-30a’, ‘hsa-mir-28’, ‘hsa- |
| | mir-25’, ‘hsa-mir-486-2’, ‘hsa-mir-182’, ‘hsa- |
| | mir-30d’, ‘hsa-mir-203a’, ‘hsa-mir-10b’, ‘hsa- |
| | mir-148a’, ‘hsa-mir-145’, ‘hsa-mir-378a’, ‘hsa- |
| | mir-143’, ‘hsa-mir-22’, ‘hsa-mir-10a’, ‘hsa-mir- |
| | 200c’, ‘hsa-mir-21’, ‘hsa-mir-192’, ‘hsa-mir- |
| | 375’ |
| Gastric | ‘hsa-mir-500b’, ‘hsa-mir-496’, ‘hsa-mir-2392’, |
| Cancer | ‘hsa-mir-5739’, ‘hsa-mir-4540’, ‘hsa-mir-6749’, |
| | ‘hsa-mir-1915’, ‘hsa-mir-202’, ‘hsa-mir-2467’, |
| | ‘hsa-mir-27b’, ‘hsa-mir-583’, ‘hsa-mir-374c’, |
| | ‘hsa-mir-219b’, ‘hsa-mir-299’, ‘hsa-mir-142’, |
| | ‘hsa-mir-30d’, ‘hsa-mir-3074’, ‘hsa-mir-147b’, |
| | ‘hsa-mir-5009’, ‘hsa-mir-624’, ‘hsa-mir-181d’, |
| | ‘hsa-mir-489’, ‘hsa-mir-581’, ‘hsa-mir-29b-2’, |
| | ‘hsa-mir-541’, ‘hsa-mir-485’, ‘hsa-mir-4519’, |
| | ‘hsa-mir-20b’, ‘hsa-mir-486-1’, ‘hsa-mir-527’ |

From the results, the number of mi-RNA biomarkers commonly in breast cancer, colon cancer, and gastric cancer was 11, and these biomarkers may be interpreted as biomarkers having common characteristics in three kinds of cancer.

TABLE 4
Biomarkers commonly in three kinds of cancer

| Kind of Cancer | Common mi-RNA Biomarker |
|---|---|
| Breast Cancer, | ‘hsa-mir-143’, ‘hsa-mir-148a’, ‘hsa-mir-182’, |
| Colon Cancer, | ‘hsa-mir-203a’, ‘hsa-mir-21’, ‘hsa-mir-22’, |
| and | ‘hsa-mir-30a’, ‘hsa-mir-30e’, ‘hsa-mir-375’, |
| Gastric Cancer | ‘hsa-mir-486-1’, ‘hsa-mir-486-2’ |

In the biomarkers common in three kinds of cancer among the biomarkers deduced from the data in Example 3 by an analysis method according to the present invention, hsa-mir-486 families were known to have a correlation with other kinds of cancer, hsa-mir-375 families were known to be circulatory biomarkers related to cancer, and hsa-mir-30 families were known to be in relation to suppression of cancer.

That is, it was confirmed that biomarkers which were previously identified as factors associated with cancer were accurately extracted as important factors by the method according to the present invention, and it may be confirmed that the result known in the art was correct.

Further, it may be appreciated that an individual cancer-specific biomarker other than the above-mentioned biomarkers is a novel individual biomarker for diagnosing cancer.

### [Example 4] Prediction of Accuracy of Biomarker Deduced Using Clinical Data

Results obtained by performing disease risk prediction calculation using the biomarker deduced by the same method as in Examples 1 and 2 were as follows.

Prediction calculation was performed so that respective measurement results of sensitivity and specificity became general results for an algorithm itself rather than results specific to a specific learning set through a 100-fold cross validation method.

A risk prediction algorithm was formed of a convolutional neural network, and composed of 7 convolutional layers and 4 fully connected layers.

All the convolutional layers were formed of a 1-dimensional filter, wherein in the first layer, a filter 20 by 1 was used, in the second layer, a filter 10 by 1 was used, and in the third layer and subsequent layers, a filter 3 by 1 was used.

In the padding, a “valid method” was used.

The fully connected layers were composed of 1024, 512, 256, and 128 nodes, and composed so that finally, a disease probability was classified using a readout layer and softmax activation.

The results are shown in Table 5.

TABLE 5
Prediction results of disease risk depending on the kind of cancer

| Kind of Cancer | Sensitivity | Specificity |
|---|---|---|
| Breast Cancer | 98.0% | 95.5% |
| Colon Cancer | 99.3% | 96.0% |
| Gastric Cancer | 99.0% | 96.2% |

From the results, in the method for predicting a disease risk based on analysis of complex genetic information according to the present invention, a biomarker having high accuracy of 95% or more was provided, and sensitivity and specificity were 95% or more. Therefore, it was confirmed that according to the present invention, an examination cost may be decreased, a commercialization possibility may be increased, and the case in which a disease risk may be confirmed by one-time examination using main genetic information associated with a large number of diseases is increased by the method according to the present invention, such that the present inventors confirmed that the method according to the present invention may be utilized as a diagnosis technology satisfying accuracy and economical efficiency enough to be commercially used, thereby completing the present invention.

The method for predicting a disease risk based on analysis of a complex genetic information network in the blood, developed according to the present invention may deduce a stable correlation with a disease from a small number of genetic information combinations and provide a genetic information correlation based on a network model by introducing a learning method. It is expected that a diagnosis technology satisfying accuracy and economical efficiency enough to be commercially used in an actual medical field by using the correlation between the genetic information and the disease deduced in the present invention will be secured.

Further, it is expected that the biomarker deduced in the present invention will be commercially used in manufacturing a medical device including a diagnosis chip and terminal and in disease diagnosis service to thereby be efficiently used in determining prognosis of a disease, and the like.

1. A method for predicting a disease risk based on complex genetic information network analysis, the method comprising:
extracting complex genetic information from specimens of a disease patient and a normal person;
comparing and analyzing the complex genetic information network to construct a complex genetic information library;
applying an optimization method or learning method to the complex genetic information library to deduce a disease state-specific biomarker; and
constructing a network model for predicting a disease risk from the disease state-specific biomarker and predicting a risk.

2. The method of claim 1, wherein the complex genetic information is expression or synthesis information of one or two or more selected from the group consisting of DNA, RNA, and proteins.

3. The method of claim 1, wherein the complex genetic information library is deduced and constructed by statistic analysis or the optimization method.

4. The method of claim 3, wherein at the time of constructing the complex genetic information library, an on/off tag capable of determining whether or not each genetic information factor has an influence on selection of a disease group by measuring a difference between an actual expression amount and a reference amount with respect to the corresponding genetic information factor is set.

5. The method of claim 4, wherein the setting of the on/off tag includes:
a) defining reference values of expression amounts in respective steps associated with an important genetic gene expression process as Th₁, Th₂, and Th₃, respectively, and defining variables as increase reference values (Th₁^{up}, Th₂^{up}, and Th₃^{up}) and decrease reference values (Th₁^{down}, Th₂^{down}, Th₃^{down}) when the genetic information expression amount is increased or decreased due to a disease, respectively; and
b) extracting genetic information which satisfies respective expression amount reference and of which the expression amount is changed due to the disease with respect to a specimen sample using the variables.

6. The method of claim 1, wherein the said method further includes: securing nucleotide sequence information of the corresponding genetic information at the time of extracting the genetic information to extract variants in DNA, RNA and protein sequences including single nucleotide polymorphism (SNP) variations including addition, deletion, or substitution of a nucleotide sequence or copy-number variations (CNVs).

7. The method of claim 1, wherein a biomarker usable in disease analysis is deduced by performing analysis of relation between the complex genetic information present in the complex genetic information library and a disease using the optimization method or learning method.

8. The method of claim 1, wherein a static disease network model is constructed based on the disease state-specific biomarker.

9. The method of claim 1, wherein in the constructing of the network model for predicting a disease risk and the predicting of the risk, a dynamic disease network model is constructed.

10. The method of claim 7, wherein the optimization method is selected from the group consisting of a simulated annealing method, a genetic algorithm, a tap search method, a simulated evolution method, and a probabilistic evolution method.

11. The method of claim 7, wherein the learning method is selected from the group consisting of a neural network and a deep learning method.

12. The method of claim 11, wherein the neural network is selected from the group consisting of a convolutional neural network (CNN) and a recurrent neural network (RNN).

13. The method of claim 1, wherein in view of accuracy, sensitivity of the network model for predicting the disease risk is 95% or more, and specificity thereof is 90% or more.

14. A disease state-specific biomarker deduced by the method of claim 1.
